(19) United States
(12) Patent Application Publication
(10) Pub. No.: US 2022/0008495 A1
(43) Pub. Date: Jan. 13, 2022

(54) APPLICATION OF PHLEGMYHEATCLEAR IN PREPARATION OF DRUG FOR TREATMENT OF ACUTE EXACERBATION OF CHRONIC OBSTRUCTIVE PULMONARY DISEASE

(71) Applicant: SHANGHAI KAIBAO PHARMACEUTICAL CO., LTD., Shanghai (CN)

(72) Inventors: Xiaoli Zhang, Shanghai (CN); Jingwei Mu, Shanghai (CN); Shaoyong Liu, Shanghai (CN); Xuehang Du, Shanghai (CN)

(73) Assignee: SHANGHAI KAIBAO PHARMACEUTICAL CO., LTD., Shanghai (CN)

(21) Appl. No.: 17/160,090

(22) Filed: Jan. 27, 2021

(30) Foreign Application Priority Data
Jul. 7, 2020 (CN) 202010647584.7

(51) Int. Cl. A61K 36/539; A61K 35/413; A61K 35/32; A61K 36/355; A61K 36/634; A61K 9/00; A61P 11/00

(52) U.S. Cl. CPC A61K 36/539; A61K 35/413; A61K 35/32; A61P 11/00; A61K 36/634; A61K 9/0019; A61K 36/355

(57) ABSTRACT

The invention discloses an application of phlegmyheatclear in preparation of a drug for treatment of acute exacerbation of chronic obstructive pulmonary disease. The present invention studies the effect of phlegmyheatclear on model of rats with acute exacerbation of chronic obstructive pulmonary disease. Results show that high dose, middle dose, or low dose of phlegmyheatclear can, during the acute exacerbation of chronic obstructive pulmonary disease, improve the lung function of the rats and the pathological damage of lung tissues of the rats in different degrees, and it is dose dependent. High dose, middle dose, or low dose of phlegmyheatclear can improve inflammatory reaction in different degrees. For the drug effects, the high dose and the middle dose of phlegmyheatclear are better than the low dose of phlegmyheatclear. Therefore, phlegmyheatclear can be used to prepare a drug for treatment of acute exacerbation of chronic obstructive pulmonary disease.

### BACKGROUND OF THE INVENTION

### 1. Field of the Invention

The invention relates to the field of use of phlegmyheatclear, and more particularly, to an application of phlegmyheatclear in preparation of a drug for treatment of acute exacerbation of chronic obstructive pulmonary disease.

### 2. Description of the Related Art

Chronic obstructive pulmonary disease (hereinafter referred to as “COPD”) is chronic bronchitis, emphysema which brings damage to your lung's air sacs (alveoli) structure, or a mixture thereof, and in which the airway from the bronchus to the alveoli is closed. Symptoms of this disease include long-term coughing with large amounts of sputum, shortness of breath due to a drop in air flow rate caused by airway obstruction, and some common respiratory infections (e.g., common cold). Such disease leads to a high morality in the world, and the morality increases due to smoking, air pollution, and the like.

The acute exacerbation of COPD has imposed burdens on the health status, the hospitalization rate, the rate of hospital readmission, and progress of COPD. The acute exacerbation of COPD is also a severe event, which is often accompanied by increased airway inflammation, increased production of mucus, and significant event of air getting trapped in the lungs. Those changes contribute to the exacerbation of one symptom of the acute exacerbation of COPD, that is, shortness of breath. Other symptoms include thicker sputum, increased amount of sputum, cough, and wheezing.

Phlegmyheatclear preparation is prepared from several components, that is, scutellaria baicalensis, bear gall powder, cornu gorais, honeysuckle flowers and fructus forsythia. It has functions of clearing heat, detoxication, resolving phlegm, spasmolysis, bacteriostasis, antiviral action, antipyresis, and immune regulation. Clinically, it can be used for the treatment of respiratory diseases, liver and gallbladder diseases, digestive system diseases etc. However, there are few reports on the application of phlegmyheatclear in preparation of a drug for treatment of acute exacerbation of chronic obstructive pulmonary disease.

### SUMMARY OF THE INVENTION

Given that the foregoing problems exist in the prior art, the present invention provides an application of phlegmyheatclear in preparation of a drug for treatment of acute exacerbation of chronic obstructive pulmonary disease.

In order to achieve the above-mentioned object, detailed technical solution is as follows:

an application of phlegmyheatclear in preparation of a drug for treatment of acute exacerbation of chronic obstructive pulmonary disease is provided.

In another preferred embodiment, the phleginyheatclear is composed of scutellaria baicalensis, bear gall powder, cornu gorais, honeysuckle flowers and fructus forsythia.

In another preferred embodiment, the drug further comprises pharmaceutically acceptable excipients.

In another preferred embodiment, a dosage form of the drug is an oral dosage form or a non-oral dosage form.

In another preferred embodiment, the oral dosage form comprises tablet, powder, granule, capsule, emulsion, syrup or spray.

In another preferred embodiment, the non-oral dosage form is an injection.

The present invention studies the effect of phlegmyheatclear on model of rats with acute exacerbation of chronic obstructive pulmonary disease. Results show that high dose, middle dose, or low dose of phlegmyheatclear can, during the acute exacerbation of chronic obstructive pulmonary disease, improve the lung function of the rats and the pathological damage of lung tissues of the rats in different degrees, and it is dose dependent. High dose, middle dose, or low dose can improve inflammatory reaction in different degrees. For the drug effects, the high dose and the middle dose of phlegmyheatclear are better than the low dose of phlegmyheatclear. Therefore, phlegmyheatclear can be used to prepare a drug for treatment of acute exacerbation of chronic obstructive pulmonary disease.

### BRIEF DESCRIPTION OF THE DRAWINGS

FIG. 1 shows electron micrographs of HE staining of lung tissues (left) and bronchus (right) in a blank control group;

FIG. 2 shows electron micrographs of HE staining of lung tissues (left) and bronchus (right) in a model control group;

FIG. 3 shows electron micrographs of HE staining of lung tissues (left) and bronchus (right) in a high dose of phlegmyheatclear group;

FIG. 4 shows electron micrographs of HE staining of lung tissues (left) and bronchus (right) in a middle dose of phlegmyheatclear group;

FIG. 5 shows electron micrographs of HE staining of lung tissues (left) and bronchus (right) in a low dose of phlegmyheatclear group; and

FIG. 6 shows electron micrographs of HE staining of lung tissues (left) and bronchus (right) in a Dexamethasone group.

### DETAILED DESCRIPTION

The present invention will now be described more fully hereinafter with reference to the accompanying drawings, in which exemplary embodiments of the invention are shown. However, the invention should not be construed as limited to the embodiments set forth herein.

This embodiment provides effects of phlegmyheatclear on model of rats with acute exacerbation of chronic obstructive pulmonary disease.

1. Experimental Materials

1.1 Phlegmyheatclear Capsule

Manufacture: Shanghai Kai Bao Pharmaceutical Co., Ltd. (Shanghai, China); batch number: 1911102 Ingredients: scutellaria baicalensis, bear gall powder, cornu gorais, honeysuckle flowers and fructus forsythia.

Clinical dosage for human: 0.06 g per 60 body weight per day

Equivalent dosage for rats, as calculated by body shape coefficient:

D rat=D human*(HI rat/HI human)(W rat/W human)2/3

D rat=D human*6.3=0.06 g/kg/d*6.3=0.38 g/kg/d

### 1.1.2 Dexamethasone Tablets

Specifications: 100 tablets with each tablet contains 0.75 mg of dexamethasone; batch number: 191067; Manufacture: Zhejiang Xianju Pharmaceutical Co., Ltd. (Zhejiang, China). Before use, take 6 tablets (total 0.6 g) and 50 ml of purified water to prepare a suspension at a final concentration of 0.012 g/ml.

Usage and dosage: oral administration. A starting dosage for adult is in a range of 0.75 mg to 3.00 mg (i.e., equivalent to 1 to 4 tablets) per time, and the drug is taken 2 to 4 times a day. Maintenance dose is about 0.75 mg (one tablet) per day. The dosage levels may be varied depending on condition.

In this experiment, 0.75 mg per time, 3 times a day.

Clinical dosage for human (body weight: 60 kg):

$$\frac{\frac{0.75 mg}{per time} * times / d}{60 kg} = 0.0375 mg / kg / d .$$

Equivalent dosage for rats, as calculated by body shape coefficient:

D rat=D human*(HI rat/HI human)(W rat/W human)2/3

D rat=D human*6.3=0.0075 mg/kg/d*6.3=0.23 g/kg/d

1.2 Experimental Animals

80 specific-pathogen-free (SPF) grade SD rats (weighing 260 g to 300 g) were used for this experiment, including 40 male rats and 40 female rats. Animal quality certificate No. 1107261911004350. The rats used in the experiments were purchased from Pengyue Experimental Animal Breeding Co., Ltd. License number: SCXK(LU) 2019-0003.

Breeding environment: SPF grade experimental animal room in IVC laboratory of the first affiliated hospital of Henan University of TCM, and License number: SYXK(YU) 2015-0005. Rats were raised under well ventilated area on a 12 h light/dark cycle at a temperature of 23±2° C., a humidity of 50%-65%. The rats were raised in separate cages in sterilized plastic boxes with free access to water, 6 to 8 animals per cage.

Feed: complete nutritional feed for experimental animals, purchased from Beijing Sibeifu Biotechnology Co., Ltd. (Beijing, China) (License No.: SCXK( [symbol] ) 2019-0010). Quality inspection report of feed nutrition proves to be qualified. The feed was under moist heat sterilization at a temperature of 121° C. for 15 minutes, dried and stored for future use.

Drinking water: purified water, home made on the experiment day.

1.3 Experimental Reagents

Hongqiqu™ flue-cured tobacco filter cigarettes (tar amount: 10 mg; smoke nicotine amount: 1.0 mg; smoke carbon monoxide amount: 11 mg; Henan China Tobacco Industry Co., Ltd.); paraformaldehyde (Tianjin Chemical Reagent Co., Ltd. (Tianjin, China)); sodium dihydrogen phosphate (Xi'an Chemical Reagent Factory (Xi'an, China)); disodium hydrogen phosphate (Luoyang Chemical Reagent Factory (Luoyang, China); EDTA-K₂ anticoagulant tube (Shanghai Institute of Chemical Reagents); absolute ethanol (Zhengzhou Paini Chemical Reagent Factory (Zhengzhou, China); IL-6, IL-10 and TNF-α ELISA kits (specifications: 96T, Wuhan Boster Bio-engineering Co., Ltd. (Wuhan, China)). CRP and SAA ELISA kits (specifications: 96T, Elabscience Co., Ltd.)

Bacteria: Klebsiella pneumoniae (KP) (strain number: 46114) was purchased from National Center for Medical Culture Collections, National Institutes for Food and Drug Control. The bacterial concentration was adjusted to 6×10⁸ CFU/ml before use.

Lipopolysaccharide (LPS): purchased from Sigma, USA, batch number: L2880.

2. Animal Experiment

2.1 Model Construction

The rats were allowed to acclimate to their surroundings for 7 days since purchased from the specific company. The rats were fed sterilized feed and allowed to free access to sterilized water. Operators regularly checked purification and water and electricity operating system to keep a quite environment. 14 rats were selected to fall into a blank control group, and the remaining rats were subjected to cigarette smoking and repeatedly infected with Klebsiella pneumoniae (KP) to establish chronic obstructive pulmonary disease (COPD) stable-phase model of rats. The method comprises the steps of: light a cigarette, making smoke concentration reach 3000±500 ppm, twice a day. The rats took a rest for at least 3 hours between the two times of being exposed to cigarette smoking, lasting for a total of 12 weeks. From 1 to 8 weeks, model of rats were instilled KP suspension (6×10⁸ CFU/ml) 0.1 ml through the nasal cavity every 5 days. Instillation via the nasal cavity: a sterilized 1 ml syringe was used to draw 0.1 ml of KP suspension; the KP suspension was instilled into the rats when the rats were breathing in, and the instillation process was done in either the left and right nostrils in an alternating way. Lipopolysaccharide (LPS) 2 mg/kg was instilled into the trachea on the first day of the 13th week to establish model of rats with acute exacerbation of chronic obstructive pulmonary disease. After successful modeling, the model of rats were randomly divided into five groups, including a model control group, a high dose of phlegmyheatclear group, a middle dose of phlegmyheatclear group, a low dose of phlegmyheatclear group, and a dexamethasone group, with 12 rats in each group.

2.2 Administration and Management

The administration was started from the 2nd day of the 13th week. The same volume of purified water (10 ml/kg/d) was administered intragastrically to rats in the blank control group and the model control group, and the other groups were received high dose of phlegmyheatclear, middle dose of phlegmyheatclear, low dose of phlegmyheatclear, and dexamethasone once a day. The administration lasted for one week (as shown in Table 1). All the rats were fasted within 12 hours before sampling of blood, however, the rats were allowed to free access to water. Blood samples were collected from caudal vein for CBC. Then the rats were anesthetized by intraperitoneal injection of 10% 1.0 ml/100 g urethane. Exposed tracheal intubation was performed. Changes of the pulmonary functions of the rats were detected by using an animal pulmonary function test system (PET). Blood was collected from the abdominal aorta, and the collected blood was placed for 2 hours and centrifuged to obtain the blood serum to detect the levels of inflammatory factors IL-6, IL-10, CRP and SAA. The rat trachea and the whole lung were taken out by thoracotomy. The right main bronchus was ligated, and the left bronchoalveolar lavage fluid (BALF) was drawn out to detect the level of inflammatory factor TNF-α; the left lung was perfused with 4% paraformaldehyde for 1 hour, and the left lung was directly fixed in the 4% paraformaldehyde for pathological examination.

TABLE 1

| | | | Equivalent | | | | |
|---|---|---|---|---|---|---|---|
| | | | dose | | | | |
| | | | for | Administration | | Administration | |
| | | | human | concentration(mg/ | Administration | volume(ml/ | Administration |
| Group | Dosage | | times | ml) | route | kg) | days |
| Blank control group | 0 | | | | Gavage | 10 | 7 |
| Model control group | 0 | | | | | | |
| High dose ofPHC | 0.76 | g/kg/ [symbol] | 2.0 | 76 | | | |
| Middle dose of PHC | 0.38 | g/kg/ [symbol] | 1.0 | 38 | | | |
| Low dose of PHC | 0.19 | g/kg/ [symbol] | 0.5 | 19 | | | |
| Dexamethasone | 0.23 | mg/kg/ [symbol] | 1.0 | 0.023 mg/ml | | | |
| group | | | | | | | |

[symbol]

indicates data missing or illegible when filed

2.3 Statistics Processing

The data was analyzed by SPSS 22.0 software. One-way analysis of variance (One-Way ANOVA) was used for comparison between groups, wherein the Least Significant Difference (LSD) method was used for the rats who met the homogeneity test of variance, and Dunnett's T3 method was used for the rats who did not meet the homogeneity test of variance. The results were described in terms of mean±standard deviation (x̅±s), and the inspection level was α=0.05.

3. Test Indicators and Results

3.1 Observation of General Condition

Observe symptoms of each group of rats, such as skin color, physical activity, mental conditions, sneezing and breathing deepening every day.

After two weeks of successful modeling, the model of rats had some symptoms of different levels of metal fatigue and decreased food intake; from week 4 to week 8, the model rats experienced yellowish and dull fur, physical and mental fatigue and trendiness to lying, shortness of breath, loose stool, and wet litter. From week 8 to week 12, shortness of breath was accompanied by gurgling with sputum, mental fatigue and trendiness to lying, small amount of secretions in mouths and noses, decreased food and water intake. After LPS was instilled into the trachea, the rats experienced obvious shortness of breath and sputum, and some rats even have symptoms of coughing, frequent scratching of nose, and sneezing. After administration, signs of improvement were shown in rats in the high dose of phlegmyheatclear group, and the middle dose of phlegmyheatclear group, such as decreased mental fatigue, decreased shortness of breath, reduced gurgling with sputum, reduced coughing and sneezing, increased water intake and physical activities; rats in the low dose phlegmyheatclear group exhibited improved metal states, increased physical activities, decreased shortness of breath; while for rats in the dexamethasone group, physical activity was increased significantly, shortness of breath was alleviated a lot, and coughing and sneezing disappeared.

Death of rats: 5 rats died during the modeling process, wherein 1 female rat died at week 10 of modeling. By anatomy, it showed that lungs had some swelling, scattered dark red plaques and lung abscess; 1 male rat died due to overdose anesthesia during the modeling process of acute exacerbation; 3 rats (2 male rats and 1 female rat) died after intratracheal instillation of LPS, and symptoms were shown in the rats, including gurgling with sputum, shortness of breath, and anatomy revealed many dark red plaques in the lungs and lung abscess. Among the 3 rats, 1 rat had symptoms of obvious lung abscess, and 1 rat had laryngeal edema. During the administration, 1 rat in the model control group died, and anatomy showed that the rat had increased bronchial mucus secretion, a dark red swollen lung, and scattered pus spots.

3.2 Pathological Examination of Lung Tissue

The left lung was fixed with 4% paraformaldehyde, dehydrated routinely, embedded in paraffin, sliced 4 μm, and stained with conventional HE. The pathological changes of the lung were observed under light microscope. Eight slices were taken from each group, and each slice was randomly selected from 6 fields of view under the light microscope. The pictures were taken with a high-definition color pathology graphic analysis system to calculate mean linear intercept (MLI) and mean alveolar numbers (MAN) to calculate alveolar size and density. The method was to draw a “ [symbol] ”-shape in the middle of each slice, measure its length (L), record the number of alveolar septum (Ns), MLI (μm)=L/Ns, and calculate the number of alveoli (Na) in each field of view, the area of each field (S), MAN (/mm2)=Na/S. Bronchial wall thickness (Wt) represented the pathological changes of the bronchus. The short diameter of the bronchus must be within the range of 100-300 μm to specify the bronchial grade. 3 long diameters (c1/c2/c3) of each bronchu and 3 short diameters (d1/d2/d3) were measured under a 400× microscope, Wt(μm)=[(c1−d1)+(c2−d2)+(c3−d3)]/(3×2).

Morphologic of the lung tissues from the observation are shown in FIGS. 1-6:

As shown in FIG. 1, the blank control group (8 rats): in each case, alveolar structure of the lung tissue is maintained normal, inflammation infiltration in the alveolar cavity is not obvious, the alveolar wall is not significantly thickened or narrowed, the structure of bronchioles at all levels is basically normal, and no obvious inflammatory cell infiltration is seen in the lumen;

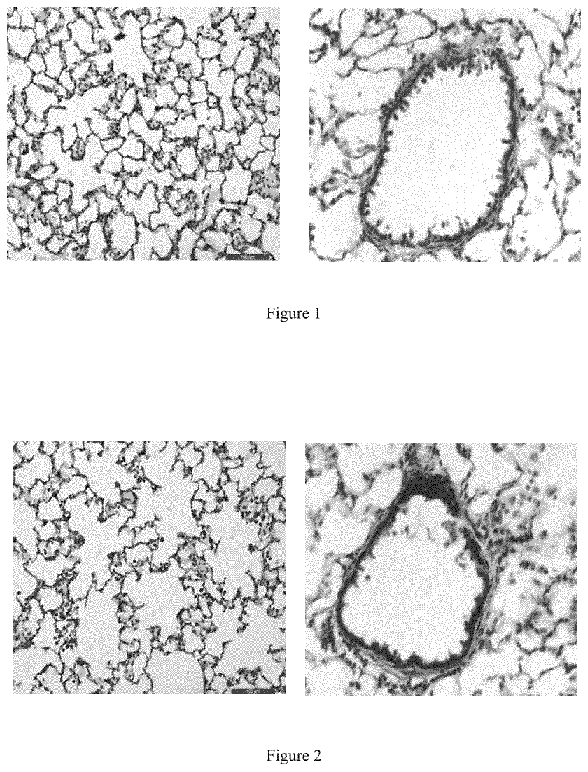

As shown in FIG. 2, the model control group (8 rats): in each case, alveolar of the lung tissue is dilated significantly, the alveolar wall is broken, parts of alveoli are fused, inflammatory cells infiltration is seen in the alveolar cavity and bronchus, the bronchial wall is thickened, infiltration of a large number of inflammatory cells is seen in the surroundings, and lumen is narrowed.

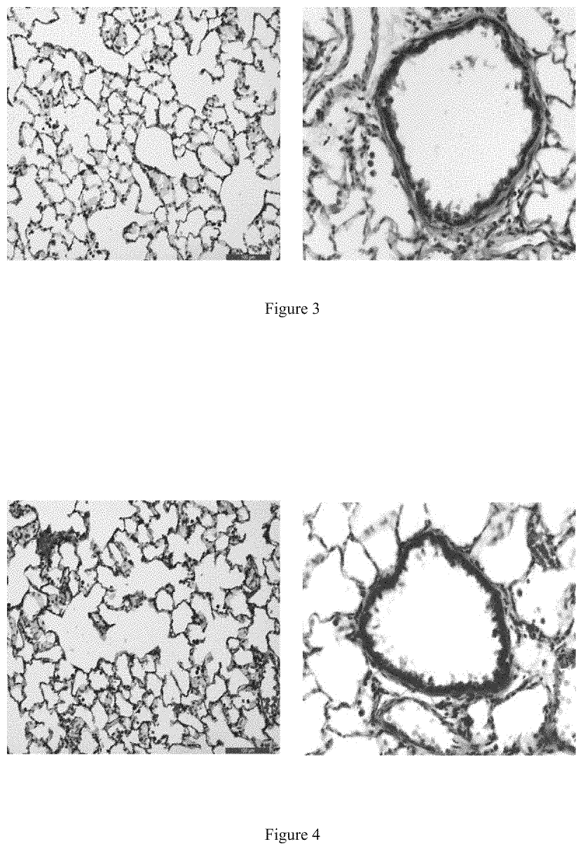

As shown in FIG. 3, the high dose of phlegmyheatclear group (8 rats): in each case, part of the alveoli is dilated in the lung tissues, infiltration of a few inflammatory cells is seen in the alveolar cavity, the alveolar wall structure is basically normal; bronchiolar epithelium at all levels is not significantly shedded, infiltration of a few lymphocytes cells is seen in the bronchial lumen, and the progress of the disease is reduced when compared with the model control group;

As shown in FIG. 4, the middle dose of phlegmyheatclear group (8 rats): in each case, part of the alveolar wall in the lung tissues is broken, infiltration of a few inflammatory cells is seen in the alveolar cavity, the structure of bronchioles at all levels is basically normal, the epithelial cells do not shed significantly, no obvious inflammatory cell infiltration is seen in the lumen, and the progress of the disease is slightly reduced when compared with the model control group;

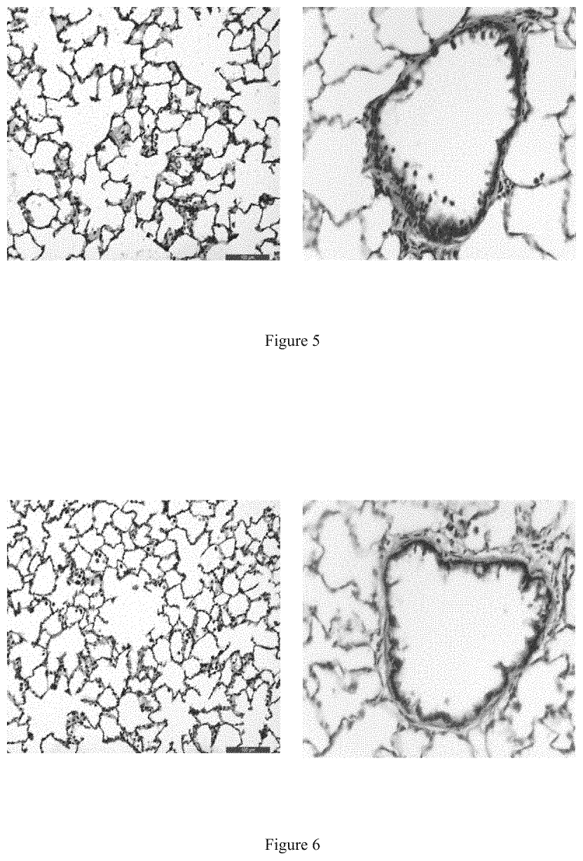

As shown in FIG. 5, the middle dose of phlegmyheatclear group (8 rats): in each case, part of the alveolar wall in the lung tissues is broken, the nearby alveoli are fused and expanded, infiltration of a few inflammatory cells is seen in the alveolar cavity and alveolar space, inflammatory cells infiltration is seen in the bronchial lumen and its surrounding areas, and the progress of the disease is not significantly reduced when compared with the model control group;

As shown in FIG. 6, the dexamethasone group (8 rats): in each case, part of the alveolar wall in the lung tissues is broken, alveolar cavity is significantly enlarged, infiltration of a few inflammatory cells is seen, the structure of bronchioles at all levels is basically normal, the epithelial cells do not shed significantly, no obvious inflammatory cell infiltration is seen in the lumen, and the progress of the disease is reduced when compared with the model control group.

For the results of the measurement of alveolar structure and bronchial wall thickness, the changes in alveolar structure and bronchial wall thickness (x̅±SD, n=8) in each group are shown in Table 2:

TABLE 2

| Group | MLI(μm) | MAN(↑/mm²) | Wt(nm) |
|---|---|---|---|
| Blank control group | 44.06 ± 6.47 | 341.13 ± 87.01 | 14.57 ± 3.37 |
| Model control | 50.84 ± 6.02 ^{a} | 276.41 ± 43.99 ^{a} | 20.14 ± 5.84 ^{a} |
| group | | | |
| Low dose group | 50.93 ± 5.64 | 265.91 ± 39.27 | 19.00 ± 2.15 |
| Middle dose group | 49.92 ± 7.16 | 296.50 ± 46.24 | 21.83 ± 4.36^{c} |
| High dose group | 46.91 ± 4.97 | 306.98 ± 62.21 | 16.43 ± 2.56 |
| Dexamethasone | 47.24 ± 5.78 | 330.49 ± 77.29 | 16.56 ± 4.01 |
| group | | | |

When compared with the blank control group, MLI in the model control group increases, MAN decreases, and the bronchial wall thickness increases (P<0.05); when compared with the model control group, MLI in the high dose of phlegmyheatclear group, the middle dose of phlegmyheatclear group and the Dexamethasone group tends to decrease, and MAN tends to increase, and there is no significant statistical difference (P>0.05): in the high dose of phlegmyheatclear group and the Dexamethasone group, the bronchial wall thickness decreases slightly (P>0.05), and the thickness of the bronchial wall in the middle dose of phlegmyheatclear group is greater than that in the Dexamethasone group (P<0.05).

3.3 Lung Functions

Before sampling, the rats were anesthetized by intraperitoneal injection of 10% 1.0 ml/100 g urethane. Exposed tracheal intubation was performed. Relevant parameters of each rat were detected by using an animal pulmonary function test system (PET), wherein the parameters include forced vital capacity (FVC), forced expiratory volume at 0.1 s (FEV0.1), forced expiratory volume at 0.3 s (FEV0.3), maximum expiratory flow rate (PEF), mid-maximum expiratory flow (MMEF), functional residual capacity (FRC), and other parameters, changes in lung function (x̅±SD) of rats in each group are shown in Table 3.

TABLE 3

| Group | FVC(mL) | FEV0.1(mL) | FEV0.3(mL) | PEF(mL/S) | MMEF(mL) | FRC(mL) |
|---|---|---|---|---|---|---|
| Blank | 15.22 ± 0.77 | 5.15 ± 1.03 | 14.31 ± 0.98 | 70.05 ± 12.11 | 69.79 ± 12.51 | 3.94 ± 1.05 |
| control group | | | | | | |
| Model group | 12.92 ± 1.74^{a} | 3.81 ± 1.13 | 12.13 ± 1.85^{a} | 61.14 ± 13.67 | 53.08 ± 11.31 | 5.36 ± 2.31^{a} |
| Low dose | 15.34 ± 2.85 ^{b} | 4.59 ± 0.83 | 14.18 ± 2.18 | 72.56 ± 12.20 | 70.00 ± 11.88 | 3.98 ± 0.93 ^{b} |
| group | | | | | | |
| Middle dose | 16.71 ± 1.88^{bb} | 4.79 ± 1.60 | 15.21 ± 1.73^{bb} | 78.11 ± 28.13 | 73.68 ± 25.31 ^{b} | 4.41 ± 0.77 |
| group | | | | | | |
| High dose | 16.11 ± 1.84^{bb} | 5.31 ± 1.59 | 15.01 ± 1.69^{bb} | 85.51 ± 17.44 ^{b} | 78.71 ± 11.82 ^{b} | 3.89 ± 1.41 ^{b} |
| group | | | | | | |
| Dexamethasone | 16.33 ± 2.01^{bb} | 5.31 ± 2.90 | 15.06 ± 2.84 ^{b} | 81.57 ± 29.43 | 78.60 ± 29.14 ^{b} | 4.37 ± 1.48 |
| group | | | | | | |

Note:
n = 6-12;
compared with the blank control group:
^{a}P < 0.05,
aaP<0.01;
compared with the model group:
^{b} P < 0.05,
^{bb}P < 0.01.

When compared with the blank control group, FVC and FEV0.3 in the model control group decreases, FRC increases (P<0.05); when compared with the model control group, FVC in the high dose of phlegmyheatclear group, the middle dose of phlegmyheatclear group, the low dose of phlegmyheatclear group and the Dexamethasone group increases (P<0.05, P<0.01), FEV0.3 and MMEF in the high dose of phlegmyheatclear group, the middle dose of phlegmyheatclear group and the Dexamethasone group increases (P<0.05, P<0.01), PEF in the high dose of phlegmyheatclear group increases, FRC in the high dose of phlegmyheatclear group and the low dose of phlegmyheatclear group decreases significantly (P<0.05).

3.4 Complete Blood Count (CBC)

Blood samples were collected from caudal vein for CBC, the following items were counted: the white blood cell count (WBC), the ratio of neutrophils (NEU %), the percentage of lymphocytes (% LYMPH) and the percentage of mononuclear cells (% MONO).

Changes (x̅±SD) of peripheral blood inflammatory cells in each group are shown in Table 4:

TABLE 4

| Group | WBC(*10⁹/L) | NEU (%) | LYM (%) | MONO (%) |
|---|---|---|---|---|
| Blank control | 4.60 ± 1.01 | 4.85 ± 4.86 | 93.13 ± 5.52 | 0.84 ± 0.49 |
| group | | | | |
| Model control | 6.73 ± 3.15^{aa} | 16.96 ± 8.73^{aa} | 81.17 ± 9.56^{aa} | 1.37 ± 1.51 |
| group | | | | |
| Low dose group | 5.27 ± 1.41 | 6.30 ± 5.03^{bc} | 92.82 ± 5.15^{bc} | 0.51 ± 0.29^{b} |
| Middle dose group | 5.22 ± 1.58 | 5.27 ± 5.46^{bbcc} | 94.09 ± 5.43^{bbcc} | 0.37 ± 0.19^{bb} |
| High dose group | 5.60 ± 1.40^{c} | 11.23 ± 15.75 | 87.78 ± 16.25 | 0.58 ± 0.41^{bb} |
| Dexamethasone | 3.80 ± 1.53^{bb} | 16.57 ± 14.99 | 82.15 ± 15.32 | 0.61 ± 0.25^{b} |
| group | | | | |

Compared with the blank control group, the peripheral blood WBC and NEU % in the model control group increases significantly, and LYM % decreases significantly (P<0.01); compared with the model control group, WBC in the dexamethasone group decreases significantly (P<0.01), NEU % in the high dose of phlegmyheatclear group, the low dose of phlegmyheatclear group decreases, LYM % increases significantly (P<0.05, P<0.01), MONO % in the high dose of phlegmyheatclear group, the middle dose of phlegmyheatclear group, the low dose of phlegmyheatclear group and the Dexamethasone group decreases significantly (P<0.05, P<0.01); compared with the dexamethasone group, NEU % in the middle dose of phlegmyheatclear group and the low dose of phlegmyheatclear group decreases significantly, and LYM % increases significantly (P<0.05, P<0.01).

3.5 Determination of Serum CRP and SAA Levels

Enzyme-linked immunosorbent assay (ELISA) is used to determine the expression of CRP and SAA in serum. Changes of serum CRP and SAA levels (x̅±SD) in each group are shown in Table 5:

TABLE 5

| Group | CRP(ng/mL) | SAA(μg/mL) |
|---|---|---|
| Blank control group | 1721.31 ± 310.64 | 12.02 ± 1.24 |
| Model control group | 2383.29 ± 514.64^{a} | 34.69 ± 11.14^{aa} |
| Low dose group | 2429.89 ± 471.75 ^{cc} | 25.66 ± 27.71 |
| Middle dose group | 2570.56 ± 487.19 ^{cc} | 25.78 ± 18.75 |
| High dose group | 2195.27 ± 419.43 ^{cc} | 24.08 ± 11.69 |
| Dexamethasone group | 3420.23 ± 1254.10^{bb} | 19.18 ± 6.18^{bb} |

Note:
n = 9-12.
Compared with the blank control group:
^{a}P < 0.05,
^{aa}P < 0.01;
compared with the model group:
bP < 0.05,
^{bb}P < 0.01;
compared with the low dose of dexamethasone group:
^{cc} P < 0.05.

Compared with the blank control group, CRP and SAA levels in serum in the model control group increases significantly (P<0.05, P<0.01); compared with the model control group, SAA level in serum in the dexamethasone group decreases significantly, and CRP levels in serum increases significantly (P<0.01); in the high dose of phlegmyheatclear group, CRP level in serum tends to decrease (P>0.05), SAA level in serum in the high dose phlegmyheatclear group, while in the middle dose phlegmyheatclear group, and the low dose phlegmyheatclear group, SAA level in serum tends to decrease. However, there is no significant statistical difference (P>0.05); compared with the dexamethasone group, CRP level in serum in the high dose of phlegmyheatclear group, the middle dose of phlegmyheatclear group and the low dose of phlegmyheatclear group decreases significantly (P<0.01).

3.6 Determination of IL-6, IL-10 in Serum and TNF-α Level in Alveolar Lavage Fluid

The expression of IL-6, IL-10 in serum and TNF-α in alveolar lavage fluid are measured by ELISA, and the results are shown in Table 6:

TABLE 6

| Group | IL-6(pg/mL) | IL-10(pg/mL) | TNF-α(pg/mL) |
|---|---|---|---|
| Blank control group | 492.94 ± 116.18 | 54.36 ± 8.74 | 360.01 ± 79.46 |
| Model control group | 855.59 ± 130.23^{aa} | 38.62 ± 7.33^{aa} | 445.86 ± 132.74^{a} |
| Low dose group | 783.98 ± 140.55^{cc} | 64.94 ± 15.66^{bbcc} | 431.33 ± 123.39^{c} |
| Middle dose group | 707.62 ± I55.18^{bcc} | 77 08 ± 13.60^{bbccd} | 330.01 ± 113.13^{bd} |
| High dose group | 652.46 ± 151.06^{bbcd} | 70.32 ± 10.11^{bbcc} | 359.78 ± 86.65^{b} |
| Dexamethasone group | 507.54 ± 185.62^{bb} | 40.65 ± 6.31 | 325.52 ± 49.51^{bb} |

Note:
n = 10-12.
Compared with the blank control group:
^{a}P < 0.05,
^{aa}P < 0.01;
compared with the model group:
^{b}P < 0.05,
^{bb}P < 0.01;
compared with the dexamethasone group:
^{c}P < 0.05,
^{cc}P < 0.01;
compared with the low dose group:
^{d}P < 0.05,
^{dd}P < 0.01.

Compared with the blank control group, TNF-α levels in the serum IL-6 and BALF in the model control group increases significantly, and the serum IL-10 decreases significantly (P<0.05, P<0.01); compared with the model control group, the serum IL-10 in the high dose of phlegmyheatclear group, the middle dose of phlegmyheatclear group, and the low dose of phlegmyheatclear group increases significantly (P<0.01), TNF-α levels in the serum IL-6 and BALF in the high dose of phlegmyheatclear group, the middle dose of phlegmyheatclear group, the low dose of phlegmyheatclear group and the Dexamethasone group decreases (P<0.05, P<0.01); Compared with the dexamethasone group, IL-6 and IL-10 levels in the high dose of phlegmyheatclear group, the middle dose of phlegmyheatclear group and the low dose of phlegmyheatclear group increases (P<0.05, P<0.01), TNF-α levels in BALF in the low dose of phlegmyheatclear group increases (P<0.05); when comparison is made among the high dose of phlegmyheatclear group, the middle dose of phlegmyheatclear group and the low dose of phlegmyheatclear group, IL-6 in the high dose group decreases more significantly that that in the low dose group (P<0.05), TNF-α levels in the middle dose group decreases when compared with the low dose group, and IL-10 increases when compared with the low dose group (P<0.05).

In conclusion, phlegmyheatclear can be used to prepare a drug for treatment of acute exacerbation of chronic obstructive pulmonary disease (COPD).

The above descriptions are only the preferred embodiments of the invention, not thus limiting the embodiments and scope of the invention. Those skilled in the art should be able to realize that the schemes obtained from the content of specification and drawings of the invention are within the scope of the invention.

What is claimed is:

1. An application of phlegmyheatclear in preparation of a drug for treatment of acute exacerbation of chronic obstructive pulmonary disease.

2. The application of claim 1, wherein the phlegmyheatclear is composed of scutellaria baicalensis, bear gall powder, cornu gorais, honeysuckle flowers and fructus forsythia.

3. The application of claim 1, wherein the drug further comprises pharmaceutically acceptable excipients.

4. The application of claim 1, wherein a dosage form of the drug is an oral dosage form or a non-oral dosage form.

5. The application of claim 4, wherein the oral dosage form comprises tablet, powder, granule, capsule, emulsion, syrup or spray.

6. The application of claim 4, wherein the non-oral dosage form is an injection.
